(19) United States
(12) Patent Application Publication
(10) Pub. No.: US 2009/0023697 A1
(43) Pub. Date: Jan. 22, 2009

(54) ANALGESIA METHOD

(75) Inventors: Colin Stanley Goodchild, Malvern (AU); Raymond Nadeson, Lethbridge (AU)

Correspondence Address: KNOBBE MARTENS OLSON & BEAR LLP, 2040 MAIN STREET, FOURTEENTH FLOOR, IRVINE, CA, 92614, US

(73) Assignee: Vernalis (R&D) Limited, Winnersh (GB)

(21) Appl. No.: 11/868,454

(22) Filed: Oct. 5, 2007

Related U.S. Application Data
(63) Continuation of application No. 10/576,515, now abandoned.
(60) Provisional application No. 60/513,097, filed on Oct. 20, 2003.

(30) Foreign Application Priority Data
Oct. 18, 2003 (GB) 0324423.3

(51) Int. Cl. A61K 31/57; A61P 25/36

(52) U.S. Cl. 514/182

(57) ABSTRACT

Opioid tolerance is reduced by treating the patient with a neurosteroid of formula (I) wherein R1 is H or methyl; R2 is OH and R3 is H, or R2 and R3 taken together are O; R4 is H or methyl; R5 and R6 are each H, or R5 and R5 taken together are O; R7 is H or methyl; and R8 is H, OH, —OC(═O)CH3, SH, —SC(═O)CH3, Cl, Br or F.

### CROSS-REFERENCE TO RELATED APPLICATIONS

This application is a continuation of and incorporates by reference in its entirety U.S. application Ser. No. 10/576,515, filed on Mar. 18, 2006, which is a U.S. National Phase of PCT International Application Number PCT/GB2004/004389, filed on Oct. 15, 2004, designating the United States of America, which claims priority under 35 U.S.C. § 119 to British Application Number 0324423.3 filed on Oct. 18, 2003.

### BACKGROUND OF THE INVENTION

1. Field of the Invention

This invention relates to the reduction of opioid tolerance in patients undergoing treatment with an opioid analgesic, for example cancer patients being treated with morphine for severe pain.

2. Description of the Related Art

Opioid analgesics, principally morphine and its derivatives, are the drugs of choice for the treatment of patients suffering severe chronic pain. Unfortunately, repeated dosing leads to tolerance and therefore reduced effectiveness. This in turn results in increasing doses of the opioid being administered, with consequential undesirable side effects. It would therefore be desirable if opioid tolerance could be reduced and the patient's level of analgesic relief restored.

### SUMMARY OF THE INVENTION

In some embodiments, a method is provided for reducing opioid tolerance in a patient undergoing treatment with that opioid who has become tolerant thereto as a result of that treatment, comprising: administering to said patient an effect amount of a compound of formula (I)

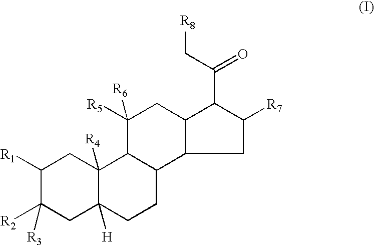

wherein
R1 is selected from the group consisting of H and methyl;
R2 is OH:
R3 is H;
or R2 and R3 taken together are O;
R4 is selected from the group consisting of H and methyl;
R5 and R5 are each H;
or R5 and R6 taken together are O;
R7 is selected from the group consisting of H and methyl;
R8 is selected from the group consisting of H, OH, —OC (═O) CH3, SH, —SC (═O) CH3, Cl, Br and F;
or a pharmaceutically acceptable derivative thereof;

said compound of formula (I) being administered either (i) while the patient is also undergoing treatment with said opioid or (ii) after cessation of treatment with said opioid and prior to resumption of treatment with said opioid.

### BRIEF DESCRIPTION OF THE DRAWINGS

FIG. 1 shows the nociceptive thresholds assessed with the ECT test for 44 hours after 250-mg/kg alphadolone administered in the slow release emulsion. Points show means and bars SEM (n=10). The nociceptive threshold was significantly increased by the one-hour reading and remained elevated for 24 hours,

FIG. 2 shows the results of the rotarod test in rats that received subcutaneous injections of emulsions of alphadolone (250 mg/kg; n=9)) and alphaxalone (80 mg/kg; n=9) given at time zero on two successive days. Alphadolone caused a small but significant amount of sedation (run time less than normal value of 120 s) at 1 hour after the injection (* p<0.05) but no sedation after that. By contrast alphaxalone caused significant sedative effects for four hours and this sedation was significantly greater than that caused by alphadolone in the first hour (* p<0.01)

FIG. 3 shows the results of the tail flick latency test in response to 6.4 mg/kg ip morphine in rats before and after they had received two day treatment with subcutaneous morphine emulsion injections in combination with subcutaneous sustained release emulsions containing alphadolone (250 mg/kg/day) or alphaxalone (80 mg/kg/day) compared with Cremophor/saline vehicle. Histograms show means and bars SEM. Significant tolerance occurred to the same extent in the vehicle and alphaxalone treated groups (* p=0.0804) whereas the tolerance that occurred in the alphadolone treated rats was less than that in saline treated controls (p=0.0101, Students t test)

FIG. 4 shows TFL responses to ip morphine (6.25 mg/kg aqueous immediate release formulation) in rats before (pre tolerance) and after (post tolerance) two days treatment with subcutaneous morphine sustained release emulsion. Intraperitoneal injection of immediate release morphine (6.4 mg/kg) on day 1 in both groups caused similar tail flick latency responses (approximately 80% MPE). Significant tolerance occurred after the two day treatment with morphine emulsion as seen on day 3 in the saline treated group (shaded histogram, p<0.01 one way ANOVA with Tukey post hoc test). By contrast the response to that same dose of morphine on day 3, when co administered with alphadolone 10 mg/kg was the same as that produced on day 1 before the treatment with morphine emulsion to cause tolerance (p>0.05, one way ANOVA). Histograms show means and bars SEM (n=9).

### DETAILED DESCRIPTION OF THE PREFERRED EMBODIMENT

U.S. Pat. No. 6,048,848 discloses the use of members of a class pregnanedione neurosteroids as non-sedating analgesics, Alphadolone (21-acetoxy-3alpha-hydroxy-5alpha-pregnane-11, 20-dione), for example as the acetate, is the exemplified member of the class. According to that publication, the compounds “may be used concurrently with other analgesic drugs to potentiate or increase the analgesic effects of those drugs” and morphine is cited as an example of such analgesics for concurrent administration.

It has now been found that the class of neurosteroids with which U.S. Pat. No. 6,048,848 may be administered to opioid-tolerant patients to reduce that tolerance, thereby restore the analgesic effectiveness of the opioid in those patients. This effect is not predictable from the disclosure of U.S. Pat. No. 6,048,848. Furthermore it is a valuable effect, since it allows the continuation of treatment with the opioid, (often the drug of choice for such patients), and restoration of its effectiveness at lower, safer doses.

Accordingly, in one aspect the present invention provides a method for reducing opioid tolerance in a patient undergoing treatment with that opioid who has become tolerant thereto as a result of that treatment, comprising treating the patient with a neurosteroid of formula (I) wherein R₁ is H or methyl; R₂ is OH and R₃ is H, or R₂ and R₃ taken together are O; R₄ is H or methyl ; R₅ and R₆ are each H, or R₅ and R₅ taken together are O; R₇ is H or methyl; and R₈ is H, OH, —OC (═O) CH3, SH, —SC (═O) CH3, Cl, Br or F; said neurosteroid being administered either (i) while the patient is also undergoing treatment with that opioid or (ii) after cessation of treatment with that opioid and prior to resumption of treatment with that opioid.

Compounds (I) may be used in any convenient solvate-, salt-, tautomeric-, analgesically active metabolite-or prodrug-form thereof. Salt forms include the acetate, sulphate, and methane sulphonate. Analgesically active metabolite forms of the compounds (I) include the glucuronide.

In the class of neurosteroids (I), R₁ is preferably H. R₂ and R₄ are preferably in the alpha conformation.

A particularly preferred compound (I) for use in the method of the invention is Alphadolone (21-acetoxy-3alpha-hydroxy-5alpha-pregnane-11, 20-dione), for example in the form of the acetate salt. Other preferred compounds (I) are 3alpha-hydroxy-5alpha-pregnane-20-one and 3alpha-hydroxy-5beta-pregnane-20-one.

In the method of the invention the neurosteroid may be administered to the opioid tolerant patient while he or she is also undergoing treatment with that opioid. In this case, doses of the neurosteroid may be administered with doses of the opioid, or alternating doses of neurosteroid and opioid may be administered. Alternatively, treatment of the patient with opioid may be stopped, and a period of dosing with the neurosteroid commenced, during which time opioid sensitivity is restored. Since the neurosteroid has analgesic activity in its own right, cessation of the opioid treatment should not cause the patient excessive discomfort as a result of his or her ongoing pain condition.

After restoration of opioid sensitivity, dosing with the opioid may be resumed, either with or without co-treatment with the neurosteroid. In the latter case however, it is to be expected that opioid tolerance will return, and the method of the invention may then be applied again to reduce that tolerance.

Dosage with the neurosteroid may be by any convenient route, using appropriate formulations. Thus, it may be formulated for intragastric (especially oral), subcutaneous, intramuscular, intravenous, or transdermal administration. An advantage of the present invention lies in the fact that administration of tolerance-reducing analgesic doses of the neurosteroid (I), such as alphadolone, by routes other than intravenous does not result in overt sedation. Hence administration non-intravenous routes will usually be preferred, especially oral administration, for example in the form of tablets or capsules, or in liquid form as solutions, suspensions or syrups.

In another aspect, the invention provides the use of a neurosteroid of formula (I) as defined and discussed above in the manufacture of a medicament for reducing opioid tolerance in an opioid-tolerant patient, especially for reducing morphine tolerance in a morphine-tolerant patient.

In the following examples, alphadolone acetate is used as a representative member of the neurosteroid class compounds with which the invention is concerned. Alphaxalone, a steroid which is not a member of the class of neurosteroids (I) with which this invention is concerned, and which is known to produce sedation, is used as a comparison compound. Morphine is used as an opioid to which patients are known to grow tolerant during long term treatment.

Examples: This work was carried out with permission of the Monash University Standing Committee on Ethics in Animal Experimentation (SCEAE Project No. MMCB 2001/15). In all experiments attention was paid to ethical guidelines for the investigation of experimental pain in conscious animals (Zimmerman 1983). Rats were housed singly with a 12 h light/dark cycle with free access to food and water.

### Preparation of Sustained Release Emulsions and Induction of Tolerance

The method of formulating morphine in a slow release emulsion has already been described (Salem and Hope 1998). An aqueous solution of morphine sulphate (David Bull Laboratories, Mulgrave, Australia) was mixed with liquid paraffin and mannide mono-oleate and stirred to form an emulsion. This method leads to sustained release of the morphine from a subcutaneous depot over a 24 hr period with none left at the subcutaneous injection site after 24 hours. Nine rats were given 1.0 ml subcutaneous injections of the morphine emulsion to induce morphine tolerance as shown:
day one am-62.5 mg/kg morphine as emulsion
day one pm-62.5 mg/kg morphine emulsion
day two am-125 mg/kg morphine emulsion

Emulsions containing alphadolone acetate (Jurox, Newcastle, Australia), alphaxalone (Jurox, Newcastle, Australia) or saline (placebo control) were also prepared. Alphaxalone was used as an active comparator since it is a neurosteroid anaesthetic very similar in structure to alphadolone but three times more potent as an anaesthetic but without antinociceptive properties. The emulsion was prepared in such a way that the neurosteroid would be released with the same time profile as had been shown for the morphine emulsion. This was tested for alphadolone by measurement of antinociceptive effects as described below. Alphadolone acetate 400 mg was dispersed in 13.6 ml 20% Cremophor EL. Saline solution (3 ml) was added to 11 ml of the alphadolone/Cremophor mix and then 8 ml liquid paraffin was added. Emulsifier (mannide mono oleate ; 1 ml) was then added to the mixture, which was stirred to produce a homogeneous emulsion. Similar emulsions were made containing vehicle only (20% Cremophor EL) and alphaxalone (133 mg).

### Characterization of Sustained Release Profile of Alphadolone Emulsion

Ten rats (wt 150-180 g) were placed in a restrainer and the nociceptive threshold was measured in the tail using the electrical current threshold test (ECT) as previously described (Edwards, Serrao et al. 1990; Serrao, Stubbs et al. 1989). Pairs of electrical stimulating electrodes were placed on the skin surface of the tail. Electrical current was passed through the electrodes (50 Hz, 1 ms pulses, 0.5 s train) to determine the minimum current necessary to cause the rat to squeak or make a strong aversive movement as defined by the up-down method. The ECT value was taken as the mean of three consecutive readings taken at 5-minute intervals after a stable baseline had been reached. The rats were then released and a subcutaneous injection of alphadolone emulsion (250 mg/kg alphadolone acetate) was given. They were returned to the restrainer for further ECT measurements at 1, 3, 6, 21, 24, 27 and 44 hours after the alphadolone injection.

Comparison of the Sedative effects of Alphadolone and Alphaxalone Sustained Release Emulsions

Sixteen male Wistar rats (wt 180-200 g) were divided into two equal groups. Each rat was subjected to the rotarod running test to assess sedation. The rats were naive to the drugs with no previous exposure to the rotarod test. They were placed on the rotarod accelerator treadmill (7650 accelerator rotarod, Ugo Basile, Italy) set at the minimum speed for two training sessions of 1-2 minutes separated by an interval of 30-60 minutes. After this conditioning period the rotarod running time was tested before the neurosteroid injection and at one-hour intervals thereafter for eight hours. Each rat received a 1.0 ml subcutaneous injection of either alphadolone (250 mg/kg) or alphaxalone (80 mg/kg) subcutaneously in a sustained release emulsion in an observer-blinded manner. At each testing time the rats were placed individually onto the rotarod set at a constant speed of 4 revolutions per minute. As the animal took grip of the drum the accelerator mode was selected on the treadmill, i.e. the rotation rate of the drum was increased linearly at the rate of 20 revolutions per minute every minute thereafter. The time was measured from the start of the acceleration period until the rat fell off the drum. A cut-off or maximum runtime for the test was 2 minutes because normal non-sedated rats all ran for 2 minutes at which time the test was terminated. The runtime values were combined for each drug and testing time to calculate means SEM. This process was repeated on the following day to mirror the protocol for administration of the neurosteroids in combination with morphine emulsion to test for their effects on tolerance as described below.

### Effect of Sustained Release Alphadolone on the Development of Morphine Tolerance

Male Wistar rats (wt 180-200 g) were treated with morphine sustained release emulsion to cause morphine tolerance as described above. They were divided randomly into two groups. In addition to the treatment with morphine emulsion, group 1 (n=27) received a 1.0 ml subcutaneous injection of emulsion vehicle with no drug added, group 2 (n=13) received a subcutaneous injection of alphadolone emulsion (250 mg/kg alphadolone acetate) and group 3 (n=15) received a subcutaneous injection of alphaxalone emulsion (80 mg/kg alphaxalone). These extra subcutaneous injections were given in the mornings of day one and day two at the same time as the morphine injections. These injections were given such that the observer making the measurements of antinociceptive effects was unaware as to the nature of the treatment that each rat had received. Shuffling a set of unlabelled envelopes containing the details of each rat and the allocated treatment randomised the treatments.

At the beginning of day one prior to subcutaneous injections, and in the morning of day three after the treatment with morphine and alphadolone as described above, each rat was placed in a restrainer. Nociceptive thresholds were measured in the tail every five minutes using tail flick latency (TFL) as described previously (Edwards, Serrao, Gent, and Goodchild 1990; Serrao, Stubbs, Goodchild, and Gent 1989). After three stable control pre drug injection readings had been obtained, an immediate release aqueous solution of morphine sulphate (6.4 mg/kg; David Bull Laboratories, Mulgrave, Victoria, Australia) was injected ip and measurements of nociceptive thresholds were continued every five minutes for 25 minutes. The antinociceptive effects were calculated as percentage maximum possible effect (% MPE) for tail flick as described previously. In this way the presence or absence of tolerance to morphine could be shown in the three groups by comparison of the responses to ip morphine on day one (pre-treatment) with the responses to the same dose on day three after the morphine emulsion treatment (post-treatment).

### Effect of Alphadolone on Established Morphine Tolerance

Two groups of rats (wt 150-299 g; n=9 each group) were treated as follows in a randomised placebo controlled fashion:
on day one TFL response was measured in each rat to an ip injection of an immediate release aqueous solution of morphine 6.4 mg/kg
all rats were then made tolerant to morphine by subcutaneous morphine emulsion injections for two days as described above
on the third day TFL responses were measured in response to a further ip aqueous morphine sulphate injection (6.4 mg/kg) given in combination with either 1.0 ml saline or a 1.0 ml saline suspension containing alphadolone 10 mg/kg. An observer unaware of the nature of this treatment, saline or alphadolone, made the TFL measurements.

The TFL responses on day 1 and day 3 were combined for drug treatment and expressed as means SEM. These values were compared to see if tolerance occurred and whether co administration of alphadolone caused reversal of the tolerance effect.

### Statistics

Replicate values for drug and time of measurement were combined and expressed graphically as means SEM. Comparisons between treatments were made with one-way ANOVA with a Tukey post hoc test. A value of p<0.05 was regarded as statistically and biologically significant.

### RESULTS

### Characterization of Sustained Release Profile of Alphadolone Emulsion

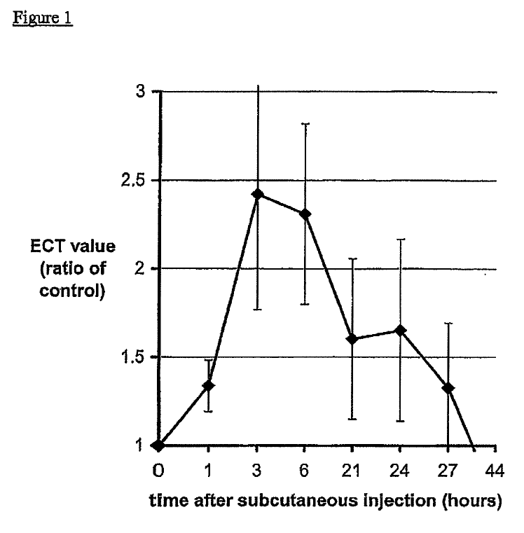

The alphadolone injections did not cause any rat to lose consciousness or in any way to look sedated. FIG. 1 shows the nociceptive thresholds assessed with the ECT test for 44 hours after 250-mg/kg alphadolone administered in the slow release emulsion. Points show means and bars SEM (n=10). The nociceptive threshold was significantly increased by the one-hour reading and remained elevated for 24 hours. Thus the emulsion slowly released the alphadolone to extend the activity for a continuous 24-hour period. Therefore the alphadolone administered in this emulsion could be expected to be present in the rat with a time profile similar to that published for the morphine emulsion (Salem and Hope 1998).

Comparison of the Sedative effects of Alphadolone and Alphaxalone Sustained Release Emulsions

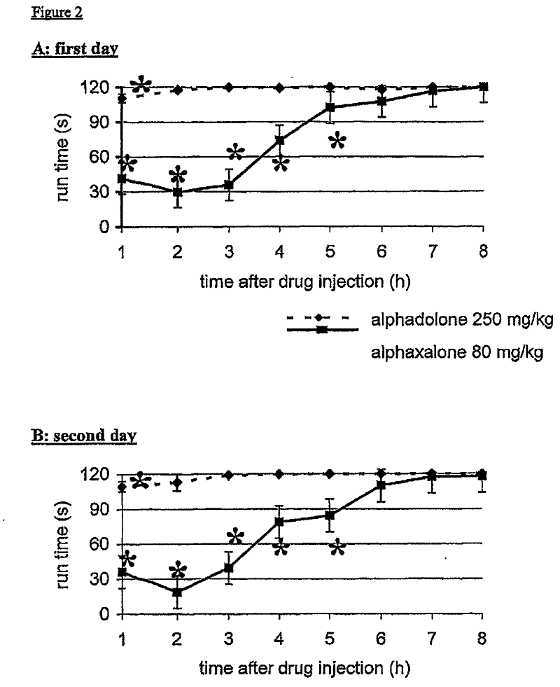

FIG. 2 shows the results of the rotarod test in rats that received <BR> <BR> subcutaneous injections of emulsions of alphadolone (250 mg/kg; n=9)) and alphaxalone (80 mg/kg; n=9) given at time zero on two successive days.

Alphadolone caused a small but significant amount of sedation at 1 hour after the injection (p<0.05) but no sedation after that. By contrast alphaxalone caused significant sedative effects for four hours and this sedation was significantly greater than that caused by alphadolone in the first hour (p<0.01)

### Effect of Sustained Release Alphadolone on the Development of Morphine Tolerance

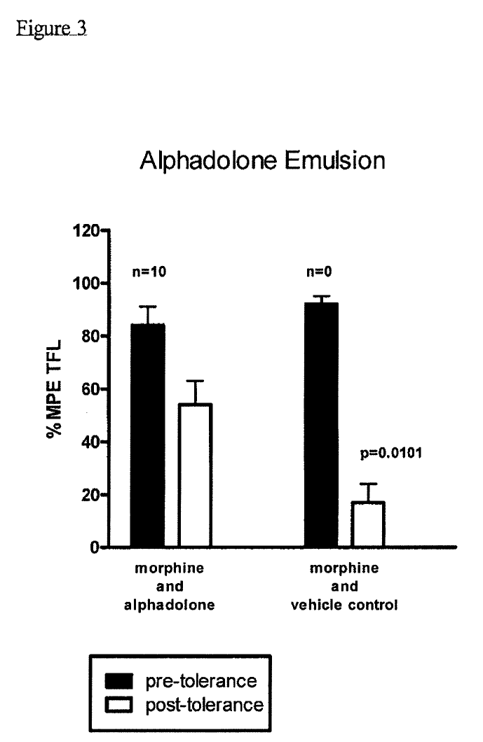

FIG. 3 shows the results of the tail flick latency test in response to 6.4 mg/kg ip morphine on days one and three in those rats that received subcutaneous morphine emulsion injections in combination with subcutaneous sustained release emulsions containing alphadolone or alphaxalone compared with Cremophor/saline vehicle. The pre treatment TFL responses to 6.4 mg ip immediate release morphine were the same for all three groups (p>0.05). Significant tolerance to morphine did occur in the vehicle treated group (p<0.001, one way ANOVA). Although there was tolerance in the alphadolone treated group, there was a statistically significant difference between the post treatment TFL values in the saline and alphadolone treated animals (p=0.0101, Students t test). By contrast, significant tolerance occurred in the alphaxalone treated rats and the post treatment values in this group did not differ from those in vehicle treated controls (p>0.05, Students t test). Furthermore those rats treated with alphaxalone were heavily sedated during the whole course of this experiment whereas the rats treated with alphadolone showed no overt signs of sedation.

### Effect of Alphadolone on Established Morphine Tolerance

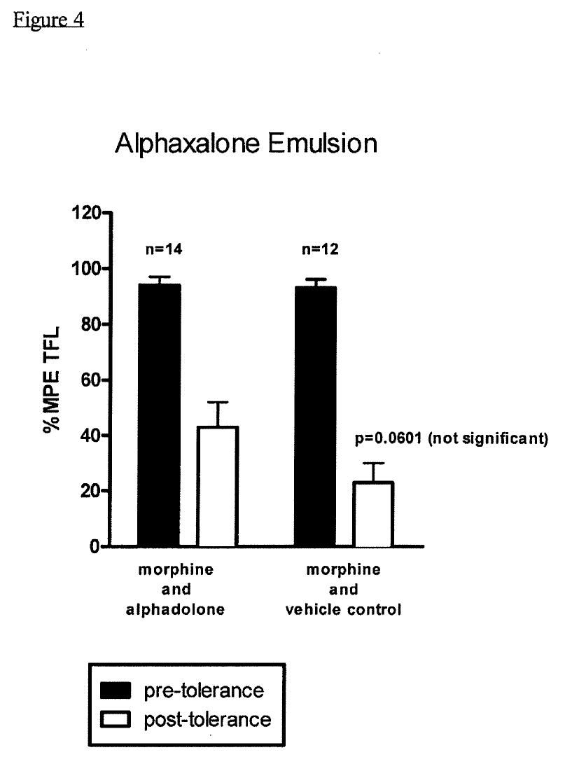

FIG. 4 shows the results of the series of experiments in which all rats received subcutaneous morphine emulsion to cause morphine tolerance. Intraperitoneal injection of immediate release morphine (6.4 mg/kg) on day 1 in both groups caused similar tail flick latency responses (approximately 80% MPE). Significant tolerance occurred after the two day treatment with morphine emulsion as seen on day 3 in the saline treated group; 6.4 mg/kg immediate release morphine given ip on day 3 in combination with saline caused only 29 8% MPE rise in TFL (p<0.01 compared with pre tolerance level; one way ANOVA with Tukey post hoc test). By contrast the response to that same dose of morphine on day 3, when co administered with alphadolone 10 mg/kg was 78.6 9.8% MPE (mean SEM) equal to that produced on day 1 before the treatment with morphine emulsion to cause tolerance (p>0.05, one way ANOVA).

### CONCLUSIONS

The conclusions to be drawn from this work are that in addition to preventing the development of tolerance to morphine when it is given at the same time as the morphine, alphadolone can also reverse the effects of established tolerance. One possible argument against such conclusions is that alphadolone is a known analgesic (Nadeson and Goodchild 2000; Nadeson and Goodchild 2001) and that the opioid tolerance and the resistance to morphine remains; that these results are merely the analgesic effect of the alphadolone alone. However, that argument does not hold because the nociceptive test that was used was TFL. Alphadolone has no antinociceptive effects in the TFL test (Nadeson and Goodchild 2000). Effective levels of alphadolone were present however. The slow release formulation containing alphadolone led to a continuous release of aiphadolone that was effective in causing antinociceptive effects revealed by the ECT test for 24 hours. The slow release morphine preparation behaved as described in the literature, causing the rats treated with this emulsion in the control group, to become tolerant; there was a marked reduction in the TFL response to morphine. By contrast rats treated with the alphadolone emulsion in combination with the slow release morphine, still responded to ip injection of morphine with a significant tail flick rise.

The next question to be asked from the results of this study concerns the selectivity of the effect. Alphaxalone was used as an active comparator. Alphaxalone and alphadolone are very similar compounds from the point of view of molecular structure and physicochemical properties (1972 ; Stock 1973). However, it has been shown by previous work that alphadolone given ip or intragastrically causes antinociceptive effects without sedation, whereas alphaxalone causes sedation and no antinociception (Nadeson and Goodchild 2000). It has been reported that alphaxalone is three times more potent as an anaesthetic compared with alphadolone. This is the reason why alphaxalone was given at the dose of 80 mg/kg compared with 250 mg/kg for alphadolone. Even so the alphaxalone still caused significant sedation compared with alphadolone. Thus, in spite of the molecular structural similarities the two neurosteroids are different pharmacodynamically as suggested by previous studies. The experiments reported in this paper also show that it is possible to use alphadolone to reverse the effects of tolerance. This effect occurred without the neurosteroid being administered while the tolerance was developing and with a dose of alphadolone that has been shown previously to potentiate morphine TFL effects (Winter, Nadeson et al. 2003).

There has been a previous report of neurosteroids preventing morphine tolerance in mice (Reddy and Kulkarni 1997). That study reported positive results for prevention of morphine tolerance for progesterone, allopregnanolone, pregnenolone sulphate and dehydroepiandrosterone sulphate. There are several significant differences between those results and the results from the experiments reported here. First, none of the compounds were found to reverse established morphine tolerance. Second, none of those compounds are pregnanediones and furthermore, unlike alphadolone, they are either not analgesics or they cause sedation and they possess hormonal properties; alphadolone is a non sedative analgesic devoid of hormonal effects.

In conclusion, the experiments reported in this paper have shown that the analgesic pregnanedione, alphadolone can prevent and reverse morphine tolerance. Alphadolone may have clinical utility as an analgesic used to enhance the antinociceptive effects of opioids by virtue of its ability to potentiate them. Therefore this result may have important clinical implications in maintaining analgesic effectiveness in long-term therapy.

### REFERENCE LIST

Steroid Anaesthesia. Glaxo Symposium on Althesin., Postgrad Med Bull, 48 (1972) 5-39.

Ben Eliyahu, S., Marek, P., Vaccarino, A. L., Mogil, J. S., Sternberg, W. F., and Liebeskind, J. C., The NMDA receptor antagonist MK-801 prevents long-lasting non-associativemorphine tolerance in the rat, Brain Res., 575 (1992) 304-308.

Edwards, M., Serrao, J. M., Gent, J. P., and Goodchild, C. S., On the mechanism by which midazolam causes spinally mediated analgesia, Anesthesiol., 73 (1990) 273-277.

Goodchild, C. S., Robinson, A., and Nadeson, R., Antinociceptive properties of neurosteroids IV: pilot study demonstrating the analgesic effects of alphadolone administered orally to humans., Br J Anaesth, 86 (2001) 528-534.

Grisel, J. E., Watkins, L. R., and Maier, S. F., Associative and non-associative mechanisms of morphine analgesic tolerance are neurochemically distinct in the rat spinal cord, Psychopharmacology (Berl), 128 (1996) 248-255.

Gutstein, H. B. and Trujillo, K. A., MK-801 inhibits the development of morphine<BR>tolerance at spinal sites, Brain Res., 626 (1993) 332-334

Houghton, A. K., Parsons, C. G., and Headley, P. M., Mrz 2/579, a fast kinetic NMDA channel blocker, reduces the development of morphine tolerance in awake rats, Pain, 91 (1901) 201-207.

Kaneko, M. and Hammond, D. L., Role of spinal gamma-aminobutyric acidA receptors in formalin-induced nociception in the rat., J Pharmacol Exp Ther, 282 (1997) 928-938.

Laurido, C., Hernandez, A., and Perez, H., Cross-tolerance to acute administration of MU and kappa opioid agonists at the spinal cord level in the rat, International Journal of Neuroscience, 87 (1996) 191-199.

Le Guen, S., Catheline, G., and Besson, J. M., Effects of NMDA receptor antagonists on morphine tolerance: a c-Fos study in the lumbar spinal cord of the rat, European Journal of Pharmacology, 373 (1999) 1-11.

Lin, Q., Peng, Y. B., and Willis, W. D., Inhibition of primate spinothalamic tract neurons by spinal glycine and GABA is reduced during central sensitization., J Neurophysiol, 76 (1996) 1005-1014.

Manning, B. H., Mao, J. R., Frenk, H., Price, D. D., and Mayer, D. J., Continuous co-administration of dextromethorphan or MK-801 with morphine: Attenuation of morphine dependence and naloxone-reversible attenuation of morphine tolerance, Pain, 67 (1996) 79-88.

Mao, J. R., Price, D. D., and Mayer, D. J., Mechanisms of hyperalgesia and morphine tolerance: A current view of their possible interactions, Pain, 62 (1995) 259-274.

Marek, P., Ben Eliyahu, S., Gold, M., and Liebeskind, J. C., Excitatory amino acid antagonists (kynurenic acid and MK-801) attenuate the development of morphine tolerance in the rat, Brain Res., 547 (1991) 77-81.

Mayer, D. J., Mao, J., and Price, D. D., The development of morphine tolerance and dependence is associated with translocation of protein kinase C, Pain, 61 (1995) 365-374.

Nadeson, R. and Goodchild, C. S., Antinociceptive properties of neurosteroids II. Experiments with Saffan and its components alphaxalone and alphadolone to reveal separation of anaesthetic and antinociceptive effects and the involvement of spinal cord, Pain, 88 (2000) 31-39.

Nadeson, R. and Goodchild, C. S., Antinociceptive properties of neurosteroids III: experiments with alphadolone given intravenously, intraperitoneally, and intragastrically., Br J Anaesth, 86 (2001) 704-708. <BR> <BR> <P>Reddy, D. S. and Kulkarni, S. K., Chronic neurosteroid treatment prevents the development of morphine tolerance and attenuates abstinence behavior in mice, Eur J Pharmacol, 337 (1997) 19-25.

Salem, A. and Hope, W., Absorption of morphine from a slow-release emulsion used to induce morphine dependence in rats, J Pharmacol. Toxicol. Methods, 40 (1998) 159-164.

Serrao, J. M., Stubbs, S. C., Goodchild, C. S., and Gent, J. P., Intrathecal midazolam and fentanyl in the rat: evidence for different spinal antinociceptive effects published erratum appears in Anesthesiology 1989 Sep; 71 (3): 482, Anesthesiol., 70 (1989) 780-786.

Stock, J. E., Advances in small animal anaesthesia, Vet Rec, 92 (1973) 351-354.

Winter, L., Nadeson, R., Tucker, A. P., and Goodchild, C. S. Antinociceptive Properties Of Neurosteroids V: A Comparison Of Alphadolone And Alphaxalone In Potentiation Of Opioid Antinociception. Anesth Analg. 2003.

In Press Zimmerman, M., Ethical guidelines for investigation of experimental pain on conscious animals, Pain, 16 (1983) 109-110.

What is claimed is:

1. A method for reducing opioid tolerance in a patient undergoing treatment with that opioid who has become tolerant thereto as a result of that treatment, comprising:
administering to said patient an effect amount of a compound of formula (I) 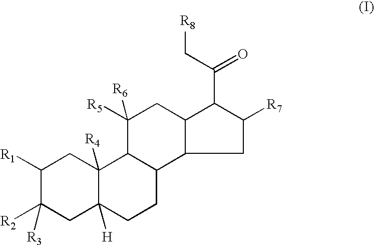
wherein
R1 is selected from the group consisting of H and methyl;
R2 is OH:
R3 is H;
or R2 and R3 taken together are O;
R4 is selected from the group consisting of H and methyl;
R5 and R5 are each H;
or R5 and R6 taken together are O;
R7 is selected from the group consisting of H and methyl;
R8 is selected from the group consisting of H, OH, —OC (═O) CH3, SH, —SC (═O) CH3, Cl, Br and F;
or a pharmaceutically acceptable derivative thereof;
said compound of formula (I) being administered either (i) while the patient is also undergoing treatment with said opioid or (ii) after cessation of treatment with said opioid and prior to resumption of treatment with said opioid.

2. The method of claim 1, wherein said compound of formula (I) is used in a form selected from the group consisting of a solvate, salt, prodrug, and analgesically active metabolite thereof.

3. The method of claim 1, wherein R1 is H.

4. The method of claim 1, wherein R2 and R4 are in the alpha conformation.

5. The method of claim 1, wherein said compound of formula (I) is selected from the group consisting of 21-acetoxy-3-alpha-hydroxy-5-alpha-pregnane-11, 20-dione, 3-alpha-hydroxy-5-beta-pregnane-20-one and 3-alpha-hydroxy-5-alpha-pregnane-20-one.

6. The method of claim 5, wherein said 21-acetoxy-3-alpha-hydroxy-5-alpha-pregnane-11, 20-dione is administered as its acetate or glucuronide.

7. The method of claim 1, wherein said opioid is selected from the group consisting of morphine, fentanyl, oxycodone, codeine, dihydrocodeine, dihydrocodeinone enol acetate, desomorphine, apopmorphine, pethidine, methadone, dextropropoxyphene, pentazocine, dextromramide, oxymorphone, hydromororphone, dihydromorphine, noscapine, papaverine, papaveretum, alfentanil, buprenorphine, tramadol and pharmaceutically acceptable derivatives thereof.

8. The method of claim 1, wherein said opioid is administered in a manner selected from the group consisting of intravenously, intramuscularly, intraperiotoneally, intragastrically, intestinally, transdermally and intrathecally.

9. The method of claim 1, wherein said compound of formula (I) is administered in an orally administrable pharmaceutical formulation.

10. A kit for reducing opioid tolerance in a patient undergoing treatment with that opioid who has become tolerant thereto as a result of that treatment, comprising:
an effective amount of a compound of formula (I) 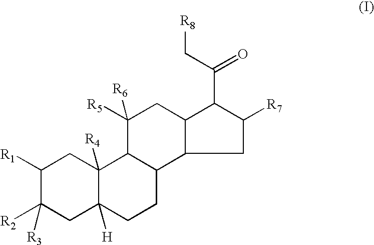
wherein
R1 is selected from the group consisting of H and methyl;
R2 is OH:
R3 is H;
or R2 and R3 taken together are O;
R4 is selected from the group consisting of H and methyl;
R5 and R5 are each H;
or R5 and R6 taken together are O;
R7 is selected from the group consisting of H and methyl;
R8 is selected from the group consisting of H, OH, —OC (═O) CH3, SH, —SC (═O) CH3, Cl, Br and F;
or a pharmaceutically acceptable derivative thereof;
instructions for administration of said compound of formula (I) either (i) while the patient is also undergoing treatment with said opioid or (ii) after cessation of treatment with said opioid and prior to resumption of treatment with said opioid in said kit.

11. A composition for reducing opioid tolerance in a patient undergoing treatment with that opioid who has become tolerant thereto as a result of that treatment, comprising:
a compound of formula (I) 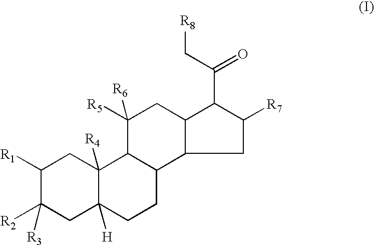
wherein
R1 is selected from the group consisting of H and methyl;
R2 is OH:
R3 is H;
or R2 and R3 taken together are O;
R4 is selected from the group consisting of H and methyl;
R5 and R5 are each H;
or R5 and R6 taken together are O;
R7 is selected from the group consisting of H and methyl;
R8 is selected from the group consisting of H, OH, —OC (═O) CH3, SH, —SC (═O) CH3, Cl, Br and F;
or a pharmaceutically acceptable derivative thereof;
said compound of formula (I) for administration either (i) while the patient is also undergoing treatment with said opioid or (ii) after cessation of treatment with said opioid and prior to resumption of treatment with said opioid.
